(19) United States
(12) Patent Application Publication
(10) Pub. No.: US 2003/0187421 A1
(43) Pub. Date: Oct. 2, 2003

(54) Container and method of sealing

(76) Inventors: Keith Manica, Lakewood, CO (US); Dennis J. Hlavinka, Arvada, CO (US)

Correspondence Address: GAMBRO, INC, PATENT DEPARTMENT, 10810 W COLLINS AVE, LAKEWOOD, CO, 80215, US

(21) Appl. No.: 10/401,818

(22) Filed: Mar. 28, 2003

Related U.S. Application Data
(60) Provisional application No. 60/368,778, filed on Mar. 28, 2002.

(51) Int. Cl. A61B 19/00; A61M 5/32

(52) U.S. Cl. 604/408; 604/412

(57) ABSTRACT

A method and apparatus for use in inactivating pathogens in fluids such as blood or blood components. The apparatus includes a container having a main body portion and at least one tubing extending into the main body portion of the container. The apparatus also includes a container having a main body portion, at least one port and tubing extending from the port into the main body portion. The method includes flowing the fluid to be inactivated into the container via the port, and sealing the port off from the main body portion of the container through the container, without sealing the main body portion of the container.

### PRIORITY CLAIM

This application claims priority from U.S. provisional application No. 60/368,778 filed Mar. 28, 2002.

### FIELD OF THE INVENTION

The present invention generally relates to the collection and storage of blood or blood components. More specifically, the present invention relates to a container which can and preferably is used in the inactivation of pathogens in blood and blood components. The container has one or more ports which are adapted to be sealed closed after the fluid to be decontaminated is flowed into the container.

### BACKGROUND

Contamination of human blood and blood components with pathogens such as human immunodeficiency virus (HIV), hepatitis and/or bacteria create a serious risk for patients who receive blood or blood components via blood transfusions. Whole blood, packed red cells, platelets and plasma (either fresh or fresh frozen) are examples of such transfusable blood and blood component products. To help combat this contamination problem, blood and biologically useful fluids can be decontaminated using pathogen inactivating agents or photosensitizers which, when activated, thereby inactivate pathogens contained in the blood or fluid but do not destroy the biological activity of the blood or fluid components.

The pathogen inactivation agents which may be useful in this invention include the class of photosensitizers known in the art to be useful for inactivating microorganisms. A “photosensitizer” as defined here is any compound which absorbs radiation of one or more defined wavelengths and subsequently transfers the absorbed energy to an energy acceptor. Such photosensitizers may be activated by the application of light to inactivate certain pathogens with which they may interact. Non-photosensitized pathogen inactivation agents are also considered within the realm of the present invention.

Various photosensitizers have been proposed for use as blood or blood component additives to inactivate pathogens in body fluids. Examples of non-endogenous photosensitizers that have been proposed for use as blood or blood component additives include porphyrins, psoralens, acridines, toluidines, flavins (acriflavin hydrochloride), phenothiazine derivatives, coumarins, quinolines, quinones, anthroquinones and dyes such as neutral red and methylene blue.

Other categories of photosensitizers are endogenous pathogen inactivation agents, such as 7,8,10-trimethylisoalloxazine (lumiflavin), 7,8-dimethylalloxazine (lumichrome), isoalloxazine-adenine dinucleotide (flavin adenine dinucleotide [FAD]), alloxazine mononucleotide (flavin mononucleotide [FMN] and riboflavin-5-phosphate), vitamin K and vitamin L and their metabolites and precursors, napththoquinones, naphthalenes and naphthols as well as their derivatives. One example of an endogenous photosensitizer contemplated for use with this invention is an alloxazine such as 7,8-dimethyl-10-ribityl isoalloxazine, commonly known as riboflavin. An advantage of using endogenous photosensitizers to inactivate blood contaminants is that endogenous photosensitizers are not inherently toxic to blood cells or to humans and if photoactivated do not yield toxic photoproducts after radiation. Therefore, no removal or purification step is required after the decontamination process, and the treated product may be stored in the same solution or portion of the solution used in the pathogen inactivation process, transfused into a patient, or returned directly to a donor's body.

Blood to be pathogen reduced may be separated into components such as plasma, platelets, white blood cells and red blood cells using the COBE Spectra™ or TRIMA® apheresis systems, available from Gambro BCT Inc., Lakewood, Colo., as well as the apheresis systems of other manufacturers. One method of decontaminating blood or blood components includes mixing an effective amount of a photosensitizer with the fluid to be decontaminated in a batch-wise way in a container; then exposing the fluid to an amount of photoradiation at an appropriate wavelength sufficient to activate the photosensitizer, causing the photosensitizer to interact with the pathogens contained within the fluid such that the pathogens contained in the fluid are inactivated. One example, not meant to be limiting are decontamination systems for use with the present invention which may be used to decontaminate previously collected and stored blood components as well as whole blood. In pathogen inactivation examples, the wavelength of light used will depend on the photosensitizing agent selected and the type of blood component being pathogen reduced. The light source or sources may provide light in the visible range, ultraviolet range, or a mixture of light in both the visible and ultraviolet ranges.

In some photosensitizer methods, the blood component to be decontaminated is flowed through an entry port into a photopermeable bag or like container. The term “photopermeable” means that the material of the container is adequately transparent to photoradiation. Either before or after the addition of the material to be decontaminated, the photopermeable container may contain a fluid used in the photoinactivation process and post-photoinactivation storage. The photoinactivation fluid may also be prepackaged in the photopermeable container.

After the pathogen inactivation process, the pathogen inactivated fluid may then flow out of the photoinactivation container into a storage container through an exit port, or may be stored in the same photopermeable container used in the photoinactivation process until transfused into a patient.

Polymeric bags and like containers are useful as photopermeable bags and such bags are typically constructed from flexible sheets of a polymeric material welded together by welds or seals along the outer border zones of the container. Such containers may in some alternative examples be blow molded. If the polymeric container is blow molded, the container would not likely have welds or seals along the outer border zones of the container.

It is known in the art to seal off externally to the container the inlets and/or outlets of a blood component container during use, such as is done with sample bags and the like, by using electromagnetic or radio frequency (RF) energy as shown for example, in U.S. Pat. No. 5,685,875 to Hlavinka. Various sorts of electromagnetic energies may be applied to create such seals, however, the choice of materials used in the container is often related to the chosen sealing method. For example, if RF energy is to be used, the container can be constructed from material that is excitable in response to RF energy. It is well known that various thermoplastic materials such as polyvinyl chloride (PVC), having high dielectric loss coefficients, may be melted by the application of RF electromagnetic fields, which excite and dielectrically heat the thermoplastic materials. U.S. Pat. No. 4,013,860, issued to Hosterman, et al for a “Hand-Held Electro-Mechanism Sealer” describes one example of an RF sealer which can be hand-held, and can be used to seal PVC or other like polymeric containers or tubes. In one embodiment, the sealer may compress the plastic material between two jaws. RF energy may then be applied to the jaws, creating an electromagnetic field, in the RF spectrum. The electromagnetic field excites and dielectrically heats the plastic material held between the jaws, partially melting the plastic material. The partially melted material welds together to form a hermetic fluid-tight seal.

It is also known in the art that materials having low dielectric loss coefficients such as polyolefins are not excitable in response to electromagnetic energy, and will not melt when exposed to electromagnetic energy. It is further known in the art that material having a low dielectric loss coefficient can be used as an energy conductant and will transfer energy through itself to a second underlying material. If the underlying material has a high dielectric loss coefficient such as PVC, the transferred energy will melt the underlying PVC material but not the overlying polyolefin material. It is against this background that the instant invention is contemplated.

A fluid container such as that used in the present invention may contain a number of ports which provide access to the interior of the container. Such ports may be manufactured, at least in part, out of semi-rigid polymeric materials which may be more rigid and thicker than the main body portion of the container. A problem with such port construction is that during viral or pathogen inactivation a portion of the fluid to be inactivated may become trapped or remain within one or more of these ports. Another problem with such ports is that they may be substantially opaque, which may prevent the passage of photoradiation through the port to the fluid contained within the port. A further problem with such ports is that fluid trapped within the ports is not exposed to the mixed contents of the container. Fluid thus trapped within the ports may still contain pathogen contaminants after the inactivation process is completed, and such contaminants may then redistribute within the otherwise inactivated fluid, thereby reinfecting the fluid.

### SUMMARY OF THE INVENTION

The invention relates to a container for containing a body fluid comprising a main body portion and at least one tubing extending both externally away from the container, and internally into the main body portion of the container.

The present invention also relates to a container for containing a body fluid comprising a main body portion, and at least a first port extending into in the main body portion of the container having a piece of tubing extending from the first port into the main body portion of the container.

The invention may also include adapting a previously existing port assembly disposed in a body fluid container comprising an intermediate component, a less rigid component, and tubing which extends into the body fluid container.

If it is desired to seal a port of a container internally to the container without forming a seal in the container, the container may be constructed of a material which is not excitable in response to electromagnetic energy. Such materials will enable an internal PVC or like material port located within a polyolefin or other non-excitable material container to be sealed closed through the container without melting the outer container material.

A further aspect of the invention includes a method of manufacturing a body fluid container. The method includes providing a main body portion of a container, providing at least one port, and extending a tube from the port into the main body portion of the container. The invention also discloses a method of retrofitting a previously manufactured container with port assemblies.

Also provided is a method of inactivating pathogens comprising the steps of flowing a fluid which may contain pathogens into a container having a main body portion, wherein at least one port has a tube extending into the main body portion of the container and sealing the tubing closed through the container to isolate the port from the main body portion of the container.

The invention will be further illuminated by the following detailed description of the invention, and brief description of the drawings. It should be noted that like elements have been given like numerals throughout the description of the invention.

### BRIEF DESCRIPTION OF THE DRAWINGS

FIG. 1 shows a plan view of a polymeric container according to one aspect of the present invention.

FIG. 2 shows a cross-sectional view of an altenative tubing portion of a container such as that shown in FIG. 1.

FIG. 3 shows a plan view of a polymeric container according to another aspect of the present invention.

FIG. 4 shows a cross-sectional view of an inlet port of the invention of FIG. 3.

FIG. 5 shows a cross-sectional view of an outlet port of the invention of FIG. 3.

FIG. 6 shows an isometric view of the inlet port of the container shown in FIG. 3 being sealed off from the main body portion of the container by a hand-held sealer.

FIG. 7 shows a plan view of a container such as that shown in FIG. 3 after being sealed closed using the hand-held sealer shown in FIG. 6.

FIG. 8 shows a series of containers utilizing a container like that shown in FIG. 3.

### DESCRIPTION OF THE PREFERRED EMBODIMENTS

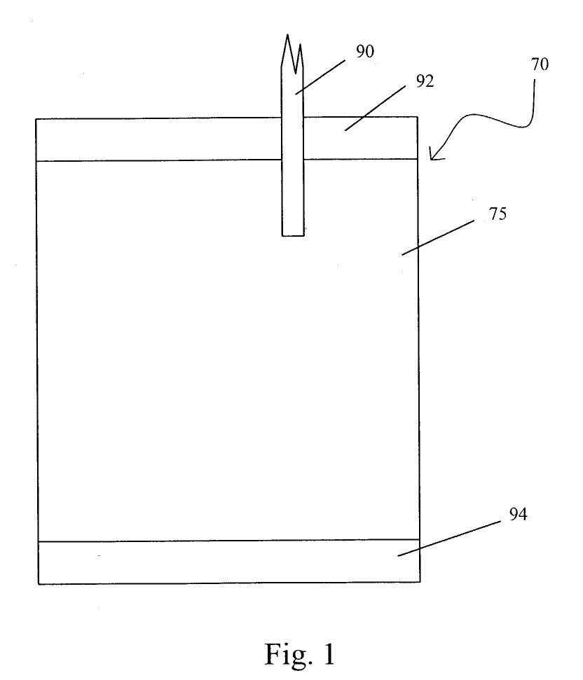

FIG. 1 shows a top plan view of a polymeric container or bag 70 which may be used in accordance with the present invention and may preferably also be used in a method or system for inactivating any pathogens or viruses in a fluid to be disposed therein. The container or bag 70 may be made from sheets of polymeric material which may be sealed or welded around the outer border zones of the container during manufacture in a continuous manner to form pre-formed seals or welds. Alternatively, as shown in FIG. 1, the container or bag 70 may be manufactured from a tube of polymeric material, necessitating seals only at the upper portion 92 and lower portion 94 of the bag. The upper and lower pre-formed seals or welds 92 and 94 create an interior space or main body portion 75 therebetween in which fluid may be contained. In one embodiment, the bag is manufactured using any material having a low dielectric loss coefficient. The material is preferably polymeric and of a polyolefin type material.

As shown in FIG. 1, a tube 90 extends through the premanufactured upper seal 92 of the bag 70 into the interior space of the main body portion 75 of the bag, to allow for fluid communication between the interior space and exterior of the container or bag. Tube 90 may extend through one of the seals of the bag as shown, or may extend into the main body portion of the bag through any other portion such as the front and/or back faces of the bag (not shown). Although only one tube 90 is shown as extending from bag 70, any number of tubes may extend from within the bag to allow for fluid transport.

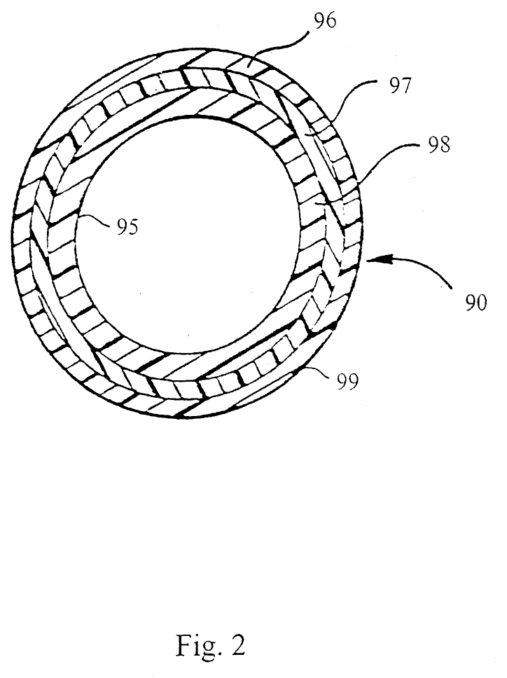

The tubing 90 may be made from a single material type in one layer of electromagnetically excitable material (as is known in the art), or as shown in greater detail in FIG. 2, the tubing 90 may be formed of a plurality, e.g., three distinct polymeric layers. The outer layer 96 of tubing 90 may be manufactured from a material having a low value for a dielectric loss coefficient. One example of a material which may be used in this capacity is a polypropylene type material although it is understood that other material having a low dielectric loss coefficient may be used. The middle or “bonding” layer 97 of tube 90 may then be composed of a material having another value for a dielectric loss coefficient. Ethylvinyl acetate (EVA) is one example of a material which may be used to form the middle layer. The innermost layer 98 may then be manufactured using a material having a high value for a dielectric loss coefficient, such as a polyvinylchloride (PVC) type material.

Tube 90 may be manufactured in a single layer embodiment by known methods, or in plural layer embodiments, by co-extruding the three polymeric materials together. Use of such a tri-layer tubing would enable the container to be manufactured without a port structure, although it may be possible to configure a single layer tubing also without a port. In particular with a plural layer tube, no port structure would be needed since the outermost surface 99 of the outer polyolefin layer 96 of tube 90 would directly bond or adhere to the polyolefin film of container 70. The innermost surface 95 of the inner PVC layer 98 of tube 90 will be capable of being sealed closed upon application of electromagnetic energy as will be more fully described below. The middle EVA layer 97 of tube 90 enables the polyolefin layer 96 and the PVC layer 98 which are unable to bond or adhere directly to one another to be formed into a single tube 90. The polyolefin layer will bond to the EVA layer which will bond to the PVC layer.

Again, it should be noted that tubing 90 could be a single layer of polymeric material depending upon the respective types of materials chosen for the bag and for the tubing.

Tubing 90 may be flexible or may be rigid. If tubing 90 is rigid, it must be relatively thin to enable it to be sealed closed using electromagnetic energy.

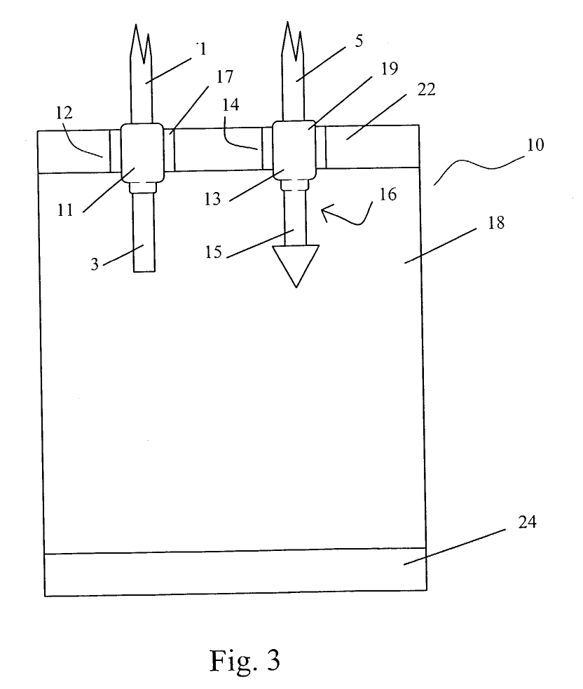

FIG. 3 is another embodiment of the present invention and may also be used in a method or system for inactivating any pathogens or viruses in a fluid contained therein. Container or bag 10 may be made from sheets of polymeric film which may be sealed or welded around the outer border zones of the container during manufacture in a continuous manner to form preformed seals or welds. Alternatively, as described with reference to FIG. 1, the container or bag 10 may be manufactured from a tube of polymeric material, necessitating seals only at the upper portion 22 and lower portion 24 of the bag.

In the embodiment shown in FIG. 3, the bag is preferably a propylflex polyolefin tri-layer co-extruded bag having a polypropylene inner layer as is currently available from Sengewald Verpackungen GmbH (Halle, Del.) However, the bag could be manufactured using any material having a low dielectric loss coefficient. The upper and lower pre-formed seals or welds 22 and 24 create an interior space or main body portion 18 therebetween in which fluid may be contained. Two ports or openings 12 and 14 extend through the pre-formed upper seal 22 of bag 10 to allow fluid ingress into and egress out of container 10. In the preferred embodiment described herein, container 10 has two ports 12 and 14; however, container 10 could have any number of ports as desired for its particular purpose, and still remain within the spirit and scope of the present invention. As shown as described herein, port 12 is an entry or inlet port which may provide for fluid ingress into the bag 10. Port 14 is an exit or outlet port which may provide for fluid egress out of the bag 10. Although shown in FIG. 3 on the upper seal 22 of the bag 10, the ports 12 and 14 respectively could be located on any of the sides of the bag without departing from the spirit and scope of the present invention. The ports 12 and 14 may also extend from any other location in the bag 10 as well.

In this embodiment, a first piece of PVC tubing 1 extends outwardly from entry port 12 in a direction away from the bag, and a second piece of PVC tubing 3 extends inwardly from entry port 12 into the main body portion 18 of the bag 10. An intermediate component 11 enables bonding or adhesion of the PVC tubing 1, 3 into ports 12, 14 and subsequently into the polyolefin container 10. Tubing 1 and 3 may also be formed from one continuous length of tubing which extends through the entire length of the inlet port 12. This single piece of tubing may be co-extruded in a manner similar to the tubing described with respect to FIG. 2. In FIG. 3, the inlet port 12 is shown in an open position to allow the fluid to be pathogen reduced to flow into the interior space 18 of the container 10.

Also in this embodiment, a third piece of tubing 5 extends out of exit port 14 away from bag 10. A frangible type connector 16 extends inwardly from exit port 14 into the main body portion 18 of the container 10. In FIG. 3 the frangible type connector 16 is shown as being attached to port 14, closing off exit port 14 and keeping any fluid contained within the bag contained.

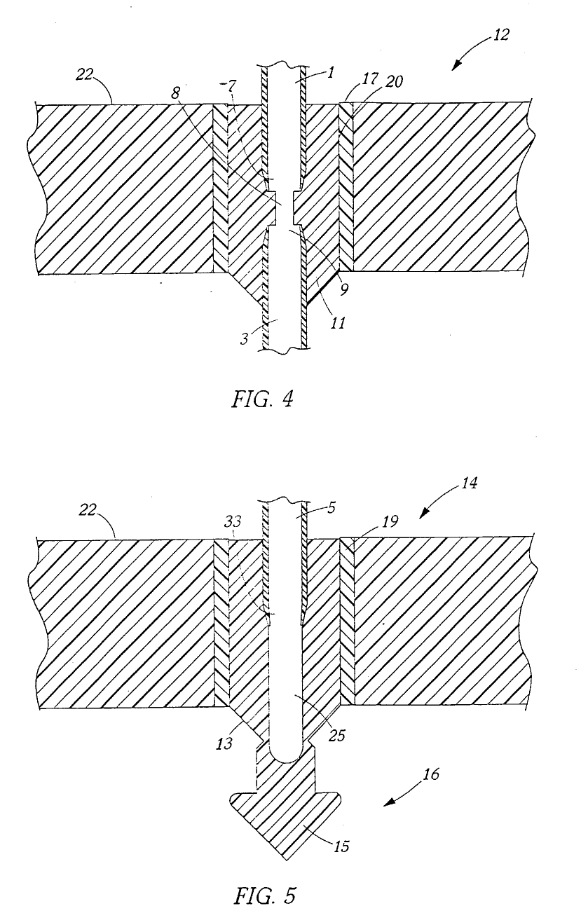

FIG. 4 shows inlet port 12 of bag 10 in greater cross-sectional detail. Port 12 includes an intermediate portion 11 which may be a bond socket having a tapered interior portion at both ends 7, 9 and a channel 8 therethrough or other structural equivalent. In this embodiment, the tapered bond socket component 11 is made of a polycarbonate material, however, any material which maintains its functional features and does not excessively deform at sterilization temperatures of 250° F. or higher and does not leach material into any fluid contained therein may be used. As shown in FIG. 4, tubing 3 extends from within one tapered end portion 9 of the bond socket component 11 of port 12 into the interior 18 of the container 10. Tubing 3 may extend any distance into the interior of the bag 18 without departing from the spirit and scope of the invention, so long as the tubing 3 is sufficiently long enough to extend a distance into the main body portion of the container to enable it to be sealed off from the main body portion 18 of the container 10 using a hand held sealer (see FIG. 6 element 65 to be described further below) through the bag. Tubing 1 also extends from within the other tapered end portion 7 of the bond socket component 11 in a direction pointing away from the bag 10. The tubing 3 which extends into the interior 18 of the bag 10, and the tubing 1 which extends away from bag 10 may be two distinct pieces of tubing which are fluidly connected to each other within the bond socket or intermediate component 11. The tubing may be flexible or may be rigid. In the presently described embodiment the pieces of tubing are made of PVC material, but other RF excitable or otherwise sealable materials may also be used.

Fluid to be pathogen inactivated may be flowed through tubing 1, through the intermediate component 11 and through tubing 3 into the interior 18 of bag 10. The outwardly pointing tubing 1 may also be used to fluidly connect the container 10 with another container such as another bag containing a blood product, or a storage bag for example.

The inlet port 12 may be manufactured in any known configuration, so long as the PVC tubing which extends into the main body portion of the bag has a means of adhering to the polyolefin bag. One type of structure that may be used either alone or in combination with another structure to both adhere tubing 3 to the bag as well as to support tubing 3 is shown by way of example as element 17 of FIG. 4. Element 17 may be a tube having a larger diameter than the diameter of the tapered bond socket component 11. In the preferred embodiment, the tube 17 is made of a two layer co-extruded polypropylene/ethylvinyl acetate (PP/EVA). The outer layer of the tube 17 is PP, which adheres or “bonds” to the polyolefin bag, while the inner layer is EVA which will “bond” to the polycarbonate intermediate component 11. The bonding may be chemical, adhesive, heat or any other type of bonding which enables polymeric materials to adhere to one another.

The inlet port 12 may also be manufactured by retrofitting a preexisting port structure in a polymeric bag. Any preexisting port structure used in any type of polymeric bag may be used, however, the polymeric material of the bag and the polymeric material of the pre-existing port must be able to adhere or “bond” to one other.

As shown in FIG. 4, the flexible or less rigid portion 17 of the port 12 may be the preexisting port structure to be incorporated therein. If a preexisting port in a bag is retrofitted, the intermediate component 11 is welded concentric with the internal end portion 20 of the preexisting port 17 before incorporation into the bag 10.

The end portions of the interior passageway of the intermediate component or tapered bond socket component 11 of the port 12 is preferably premanufactured with at least one tapered interior portion, more preferably two. The interior passageway through the tapered bond socket allows for fluid to flow in a continuous manner through the length of the port. The tapered interior portions of the bond socket create a tight fit between tubing pieces 3 and 1 respectively and the walls of the bond socket, to create a fluid tight seal between tubing pieces 3 and 1 and to hold tubing pieces 3 and 1 in place. The tapered bond sockets act to stretch the interior diameter of the flexible port portion 17 to prevent formation of gaps during manufacture or during use between intermediate component 11 of the port and flexible portion 17 to prevent any fluid leaks through the resulting gaps. It should be noted however that gap-filling measures such as use of adhesives may also be used without departing from the spirit and scope of the invention.

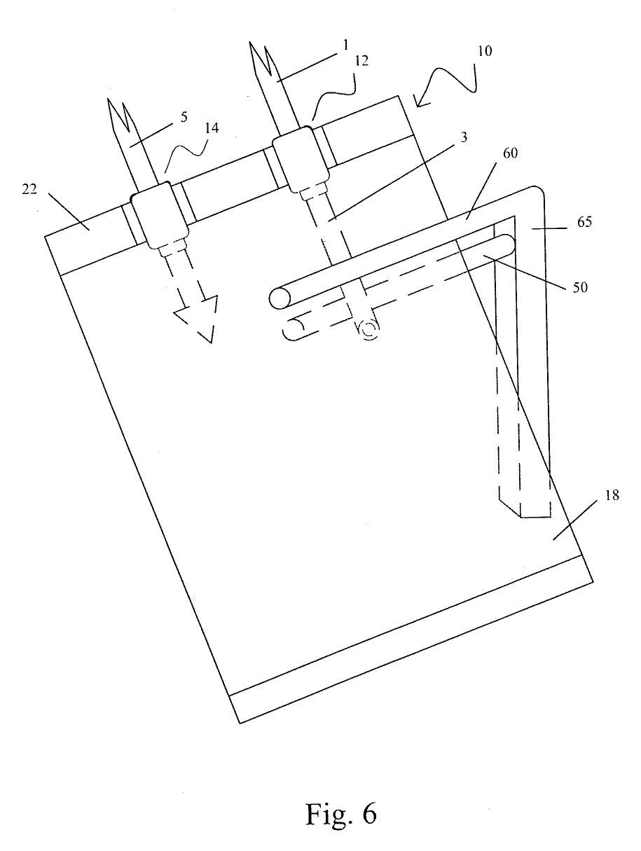

As shown in greater detail in FIG. 5, and as set forth with reference to FIG. 3, outlet port 14 preferably contains two components, a more rigid frangible component 16 and a less rigid component 19. Although a frangible component is shown as being part of the outlet port 14, the only requirement of the outlet port is that it is initially closed to contain fluid introduced into the bag via the inlet port. Other types of mechanisms capable of being selectively opened may also be used.

The frangible component 16 has two portions, an upper body portion 13 and a lower frangible pin portion 15. A channel 25 extends through the length of the upper body portion 13. Numerous such frangible connection mechanisms are known in the art. Rupturing the frangible mechanism allows the lower frangible pin portion 15 of the mechanism to be separated from the upper body portion 13 of the mechanism. Such separation opens the channel 25 in the port, thereby permitting fluid to flow through channel 25, through outlet port 14, through flexible tubing 5 and out of the container 10. Examples of frangible mechanisms that could be used in this invention are described in detail in U.S. Pat. Nos. 4,340,049 to Munsch and 5,330,464 to Mathias, although it is understood that other well known frangible connectors could alternatively be used. In one embodiment, the frangible component is available from Gambro Dasco (Mirandola, IT). In this embodiment, the more rigid frangible component 16 is made of a polycarbonate material. Any material other than polycarbonate may be used however so long as the material maintains its functional features and does not excessively deform at sterilization temperatures of 250° F. or higher and does not leach material into any fluid contained therein. Although in the described embodiment a frangible mechanism 16 is used, any means for maintaining sterility of the container as well as for allowing selective access between the interior of the container and the exterior of the container may be used.

Tubing 5 extends outwardly away from bag 10 from within a tapered bond socket 33. The outwardly pointing tubing 5 may be used to drain any fluid contained within the bag out of the bag 10 via port 14, or to fluidly connect the container 10 with another container (not shown). In one embodiment the piece of tubing 5 is made of PVC material.

The outlet port 14 may be manufactured in any known configuration, so long as the outlet port is initially closed to contain fluid introduced into the bag via the inlet port.

In a manner similar to that described above for the inlet port 12, the outlet port 14 may also be manufactured by modifying or retrofitting a preexisting port structure. Any preexisting port structure may be used, however, in one embodiment, the preexisting port structure used in a Sengewald bag could be modified to create a new port. In FIG. 5, the less rigid component 19 is the preexisting port. In this embodiment, the preexisting port structure is premanufactured from a PP/EVA type material. To modify the preexisting port 19, the polycarbonate frangible component 13 is welded concentrically to the preexisting port structure 19. In another method of retrofitting the preexisting port, the frangible portion may be inserted through the preexisting port portion.

To insert the polycarbonate components 11 and 16 of ports 12 and 14 into the preexisting portions 17 and 19 of ports 12 and 14, the interior diameter of the preexisting port structures 17 and 19 may be “pre-wetted” with cyclohexanone prior to sliding the polycarbonate components 11 and 16 into port structures 17 and 19 within preexisting ports 12 and 14. Tubing piece 1 is either previously or then inserted into the upper tapered interior portion 7 of the rigid portion 11 of port 12. Tubing piece 3 is either previously or then inserted into the lower tapered portion 9 of the rigid portion 11 of port 12. Tubing piece 3 may also be a continuous portion of tubing piece 1 and thus inserted therein with tubing piece 1.

Tubing piece 5 is inserted into the tapered portion 33 of the rigid portion 13 of port 14.

The ports 12 and 14 may also be made without using a preexisting port structure. The ports may just be intermediate components 11 or 16 or other well known port structure. The ports 12 and 14 may also be made concurrently with bag 10.

As described with reference to FIG. 3, but equally applicable to FIG. 1, in one method of using the present invention, fluid to be pathogen inactivated flows via a continuous fluid flow path from tubing 1 through inlet port 12, through tubing 3 and into bag 10. However, it should be noted that the fluid to be inactivated may flow into bag 10 by any other means not specifically described. Once fluid to be inactivated is contained within bag 10, the inlet port 12 may be sealed off from the main body portion 18 of the bag 10 by sealing tubing 3 closed. Tubing 3 is sealed off from the main body portion of the container in such a manner so as to eliminate the possibility of any stagnant fluid pockets which were inadequately reduced of pathogens contained within the ports from recontaminating the inactivated fluid, through use of a hand-held RF sealer. One or more such seals may be made in tubing 3.

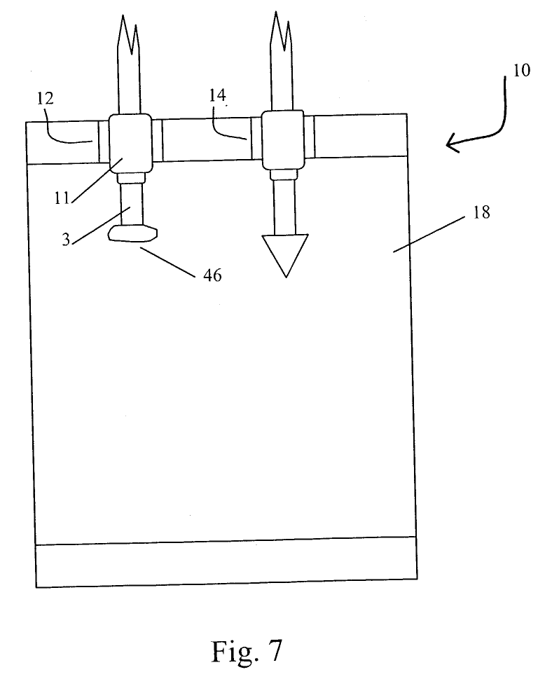

FIG. 6 is an isometric view of the container 10 of FIG. 3 with jaws 60 and 50 of a hand held heat sealer 65 being placed over tubing 3 to seal the fluid communication of the tubing 3 off from the main body portion 18 of the container 10 through the bag once fluid had been introduced. The bag 10 is shown as being flipped over from the position of the bag shown in FIG. 3, for ease of sealing. It should be noted that due to the low dielectric loss coefficient of the bag material, a seal in bag 10 will not be formed even though the hand held sealer is placed directly over the bag. The bag may also be folded over on itself (the axis of the bag folded co-linearly with port 12) which would enable tube 3 to be sealed closed no matter where tube 3 is located along bag seal 22. In other words, port 12 does not have to be located near an edge of the bag such as depicted in FIG. 6. The internal tubing 3 is shown as being longer than may be desired in practice, primarily for ease in depicting isometrically the method of sealing tube 3 using a hand-held sealer. It is only necessary that the tubing 3 extend sufficiently into the main body portion 18 of the bag to enable the tube 3 to be sealed and isolated. The tubing 3 should be sealed in such a way so as to sufficiently seal off any stagnant volume of fluid located in the interior diameter of tube 3 to prevent fluid communication with the contents of container 10. The tubing may be sealed once as shown in FIG. 7 or may be sealed multiple times (not shown.)

To seal off the port 12 from the main body of the container, the upper jaw 60 and the lower jaw 50 of the sealer 65 are placed on top of the bag and under the bag over the location in the bag where tubing 3 extends from port 12. The upper and lower jaws 60 and 50 in one embodiment are moved towards each other until they come into contact with the exterior surface of the bag 10. The jaws 60 and 50 compress both the bag 10 and the tubing 3 until the tubing 3 is squeezed together, interrupting fluid communication between the entry port 12 and the interior of the container 18. Electromagnetic energy is then applied between the jaws 60 and 50 respectively, to create an electromagnetic energy field between the jaws. The electromagnetic energy field causes dielectric heating and resultant melting of the tubing 3 to create a welded seal thereacross. When jaws 60 and 50 respectively of the sealer 65 are released from the container after the application of RF energy, a seal 46 (see FIG. 7) will have been formed in the tube 3. The seal 46 (or multiple seals) isolates the port 12 from the main body portion 18 of the container 10 and prevents fluid communication between the entry port 12 and the main body portion 18 of the container 10. In the preferred embodiment, bag 10 is made of a polyolefin or other substantially non-RF excitable material. The substantially non-RF excitable nature of the polyolefin or other like material is not melted by the application of electromagnetic energy generated by the hand held sealer. However, the RF excitable PVC tubing 3, which extends into the bag 10 from port 12 is melted by the electromagnetic energy, thus enabling the sealing of tubing 3 through the bag, but not of the surrounding bag 10.

A representative seal 46 made by such a sealer is shown in FIG. 7. Sealing off the tubing 3 which extends from the entry port 12 into the main body portion 18 of the container 10 is desirable, particularly in viral or photosensitizing methods as it separates or isolates the port 12 containing fluid which may not have been pathogen inactivated from the main body portion 18.

One hand-held sealer which may be used to form a fluid tight seal is a hand-held radio frequency (RF) welder such as the type manufactured by Sebra Company, Tucson, Ariz. However, use of any type of electromagnetic or heat sealer by any known manufacturer is contemplated to be within the scope of this invention. It is also contemplated that the electromagnetic or heat sealer can be stationary rather than hand-held such as those sealers used in known container sealing and manufacturing processes. If heat sealing is used, it should be noted that the melt temperature of the tube 3 must be lower than the melt temperature of the main body portion 18 of the bag 10.

While the fluid inside the main body portion 18 of the container or bag 10 is being inactivated, the exit port 14 remains isolated from the main body portion 18 of the container 10 by the frangible mechanism 16 of port 14. The frangible mechanism 16 allows the fluid contained in the interior 18 of the container 10 to flow out of the container only after the frangible pin portion 15 of the frangible mechanism 16 is broken, as is known and/or described above. Once the fluid has been pathogen reduced, the operator can rupture the frangible mechanism 16 by manipulatively bending the frangible pin portion 15 of the mechanism 16 to separate it from the upper body portion 13 and thereby allow the fluid to flow out of the container 10 through the passageway 25 (see FIG. 5) and exit port 14 and out external tubing 5.

Without the ability to construct a port so that it is capable of being separated from the main body portion of the bag such as the ports described above, fluid which was not adequately pathogen reduced may become trapped in stagnant pockets of fluid within the ports. The presence of stagnant pockets of fluid trapped inside the ports may prevent the photosensitizing agent in the fluid from coming into inactivating interaction with any pathogens contained within the stagnant fluid pockets, and/or may prevent photoradiation from reaching any fluid trapped therein.

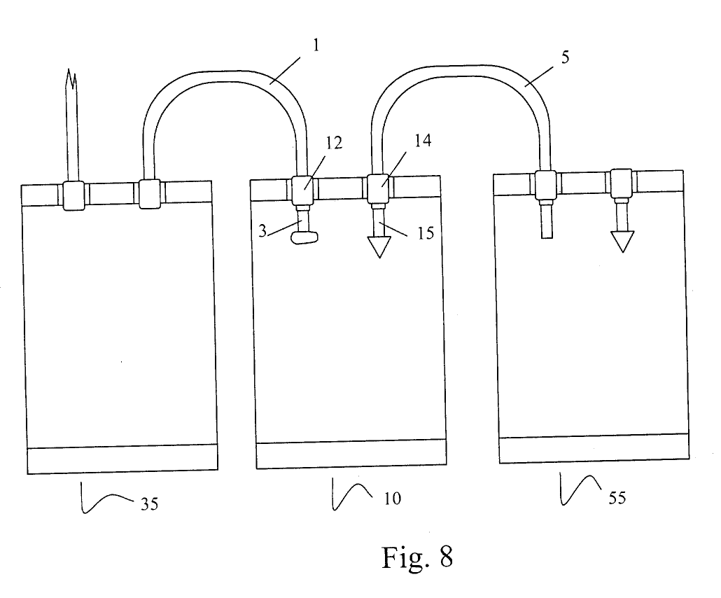

One further method of using a container 10 as described above, is as follows with reference to FIG. 8. Initially, fluid which is to be pathogen inactivated may be flowed into bag 10 from a blood component bag 35. Fluid flows from bag 35 through tubing 1, entry port 12, and tubing 3, prior to RF sealing, into bag 10. The fluid to be inactivated may be mixed with a photosensitizer before being transferred into bag 10, or the photosensitizing agent may be preliminarily disposed in bag 10 before receipt of the fluid to be pathogen inactivated, or it may be added after the fluid to be pathogen inactivated. The fluid to be inactivated and the photosensitizing agent may also be mixed within the container 10 itself, before and/or during irradiation. After the fluid to be inactivated and the photosensitizer are combined within bag 10, inlet port 12 is sealed off as is shown, from the main body portion of the container 18 by a welder such as those described above. The fluid contained within bag 10 is then exposed to light (not shown) to activate the photosensitizer contained within the fluid. Bag 10 may or may not be agitated as well to mix the components contained within the bag. After the fluid has been inactivated, the frangible pin mechanism 15 of the outlet port 14 is ruptured, allowing the inactivated fluid to flow out of container 10 via tubing 5 and into another bag 55 which may be used for storage. It should be noted however, that FIG. 8 is merely one possible use of bag 10. Other modifications are equally possible. As one example, the inactivated fluid may be collected and/or stored in the same container in which photoinactivation took place.

It should be understood that various changes and modifications to the presently preferred embodiments described herein will be apparent to those skilled in the art.

1. A container for containing a body fluid comprising:
a main body portion to contain the body fluid; and
at least a first piece of tubing wherein the first piece of tubing provides fluid communication between the interior of the main body portion and the exterior of the container.

2. The container according to claim 1 further comprising at least a first port disposed in the main body portion.

3. The container according to claim 2 wherein the first piece of tubing extends from within the first port into the interior of the main body portion.

4. The container according to claim 2 wherein the first port further comprises a second piece of tubing which extends from within the first port in a direction external to the container.

5. The container according to claim 4 wherein the first or second piece of tubing is flexible.

6. The container according to claim 4 wherein the first or second piece of tubing is rigid.

7. The container according to claim 4 wherein the first and second pieces of tubing enable the body fluid to flow into interior of the main body portion via the first port.

8. The container according to claim 1 wherein the first piece of tubing is comprised of a material capable of forming a seal in response to electromagnetic energy.

9. The container according to claim 1 wherein the first piece of tubing is comprised of a high dielectric loss material.

10. The container according to claim 9 wherein the first piece of tubing further comprises PVC-type material.

11. The container according to claim 1 wherein the first piece of tubing further comprises an inner layer of a material having a high dielectric loss coefficient and an outer layer of a material having a low dielectric loss coefficient.

12. The container according to claim 11 wherein the inner layer of material further comprises polyvinylchloride.

13. The container according to claim 11 wherein the outer layer of material further comprises polyolefin.

14. The container according to claim 11 wherein the first piece of tubing further comprises a middle layer of material between the inner layer and the outer layer.

15. The container according to claim 14 wherein the middle layer of material further comprises ethylvinylacetate.

16. The container according to claim 14 wherein the first piece of tubing is co-extruded.

17. The container according to claim 1 wherein the main body portion is photopermeable.

18. The container according to claim 1 wherein the main body portion comprises a material not directly capable of forming a seal in response to electromagnetic energy.

19. The container according to claim 1 wherein the main body portion further comprises a low dielectric loss material.

20. The container according to claim 19 wherein the main body portion further comprises a polyolefin-type material.

21. The container according to claim 1 further comprising a second port wherein the second port has a frangible portion.

22. The container according to claim 21 further comprising a third piece of tubing which extends from within the second port in a direction external to the container.

23. The container according to claim 1 wherein the first piece of tubing and the main body portion are made of material such that the first piece of tubing is capable of being sealed closed through the main body portion of the container without sealing the main body portion of the container by application of electromagnetic energy to the main body portion and tubing.

24. A port assembly adapted to be disposed in a body fluid container comprising;
an intermediate component;
a less rigid component concentric to the intermediate component; and
a piece of tubing;
wherein the tubing is adapted to extend from within the intermediate component into the interior portion of the body fluid container.

25. The port assembly according to claim 24 wherein the intermediate component comprises a material which is capable of bonding to a material having a high dielectric loss coefficient.

26. The port assembly according to claim 25 wherein the intermediate component further comprises a polycarbonate material.

27. The port assembly according to claim 24 wherein the piece of tubing is flexible.

28. The port assembly according to claim 24 wherein the piece of tubing is rigid.

29. The port assembly according to claim 24 wherein the piece of tubing is further comprised of a material having a high dielectric loss coefficient.

30. The port assembly according to claim 24 wherein the piece of tubing is further comprised of a PVC-type material.

31. The port assembly according to claim 24 wherein the piece of tubing is capable of being sealed closed through the body fluid container without sealing the body fluid container by application of electromagnetic energy to the body fluid container and the tubing.

32. The port assembly according to claim 24 further comprising a second piece of flexible tubing which is adapted to extend from within the intermediate component in a direction external to the container to provide for fluid flow through the port assembly into the body fluid container.

33. A method of manufacturing a body fluid container comprising:
providing a main body portion of a container;
providing at least a first port; and
extending a piece of tubing from within the first port into the main body portion of the container.

34. A method of retrofitting a previously manufactured container for containing body fluid comprising:
providing a previously manufactured main body portion of a container having a previously manufactured port;
bonding an intermediate component concentric to the previously manufactured port to create a first port; and
bonding tubing into the intermediate component of the first port so that it extends into the main body portion of the container.

35. The method according to claim 34 further comprising providing a second previously manufactured port; and
bonding a relatively rigid frangible component concentric to the second previously manufactured second port to create a port.

36. A method for inactivating pathogens in a fluid which may contain pathogens comprising the steps of:
flowing a fluid which may contain pathogens into a container having
a main body portion;
at least one tubing which extends into the main body portion of the container; and
sealing the tubing closed through the main body portion of the container without sealing the main body portion of the container to isolate the tubing from the main body portion of the container.

37. The method according to claim 36 wherein the step of flowing further comprises flowing the fluid into the container having at least one port having a frangible mechanism.

38. The method according to claim 36 wherein the step of flowing further comprises adding a pathogen inactivation agent to the fluid to be pathogen inactivated.

39. The method according to claim 38 further comprising inactivating the fluid by exposing the fluid and pathogen inactivation agent to light of an appropriate wavelength to inactivate pathogens contained in the fluid.

40. The method according to claim 38 further comprising the step of breaking the frangible mechanism to open the port in the container, allowing the inactivated fluid to flow out of the container.

41. The method according to claim 36 wherein the step of sealing the tubing closed through the main body portion of the container without sealing the main body portion of the container further comprises;
aligning the tubing between an upper jaw and a lower jaw of a sealer;
compressing the jaws of the sealer; and
applying energy to create a seal.

42. The method according to claim 41 wherein the step of applying energy to create a seal comprises applying radio frequency electromagnetic energy.

43. The method of claim 41 wherein the step of applying energy comprises applying heat energy.
